(19) United States
(12) Patent Application Publication
(10) Pub. No.: US 2008/0300556 A1
(43) Pub. Date: Dec. 4, 2008

(54) VIEWABLE COVER FOR AN OSTOMY POUCH

(75) Inventor: Gary H. Fenton, Pepper Pike, OH (US)

Correspondence Address: PEARNE & GORDON LLP, 1801 EAST 9TH STREET, SUITE 1200, CLEVELAND, OH, 44114-3108, US

(73) Assignee: Marlen Manufacturing and development Co., Inc., Bedford, OH (US)

(21) Appl. No.: 12/130,169

(22) Filed: May 30, 2008

Related U.S. Application Data
(60) Provisional application No. 60/941,311, filed on Jun. 1, 2007.

(51) Int. Cl. A61F 5/44

(52) U.S. Cl. 604/339

(57) ABSTRACT

An ostomy drainage receptacle comprising a flexible pouch defined by a pair of walls. A stoma receiving opening in a first one of the walls. A second one of the walls being translucent or transparent. A third opaque wall sealed along a portion of its edges to the second one of the walls. A fourth opaque wall sealed along a portion of its edges to the second one of the walls and slightly overlapping an edge of the third wall.

This application claims the benefit of U.S. Provisional Application No. 60/941,311, filed on Jun. 1, 2007, the entire disclosure of which is hereby incorporated herein by reference.

This invention relates to ostomy appliances and,

more particularly to an ostomy pouch having its distal panel covered by a cover comprising an opaque or translucent panel. The cover is openable to permit the user to view the condition of the stoma.

Ostomy pouches are either transparent or opaque. Opaque pouches are preferred by many who wish to conceal the contents of the pouch. This is important when wearing thin, summer clothing. On the other hand, transparent pouches permit the user to inspect the condition of his or her stoma without removing the pouch. Since both pouches have desirable attributes, it would be desirable to provide an ostomy pouch that would mask the contents of the pouch but permit the user to inspect the condition of the stoma without removing the pouch.

### SUMMARY OF THE INVENTION

This invention provides an ostomy pouch which masks the contents of the pouch and permits the user to inspect the stoma without removing the pouch. This is accomplished by providing a first opaque or translucent panel on the distal panel of a translucent pouch and extending from about the bottom of the pouch to a level at approximately the level of the stoma opening in a proximal panel of the pouch. A second opaque or translucent panel extends from the top of the pouch and overlaps the first panel. The overlapping relationship of the first and second panels allows the user to part the panels slightly to inspect the stoma.

### BRIEF DESCRIPTION OF THE DRAWINGS

FIG. 1 is a proximal elevational view of an ostomy appliance according to this invention;

FIG. 2 is a cross-sectional view, the plane of the section being indicated by the line 2-2 in FIG. 1;

FIG. 3 is a distal elevational view of an ostomy appliance according to this invention; and

FIG. 4 is a cross-sectional view, the plane of the section being indicated by the line 4-4 in FIG. 3.

### DETAILED DESCRIPTION OF THE INVENTION

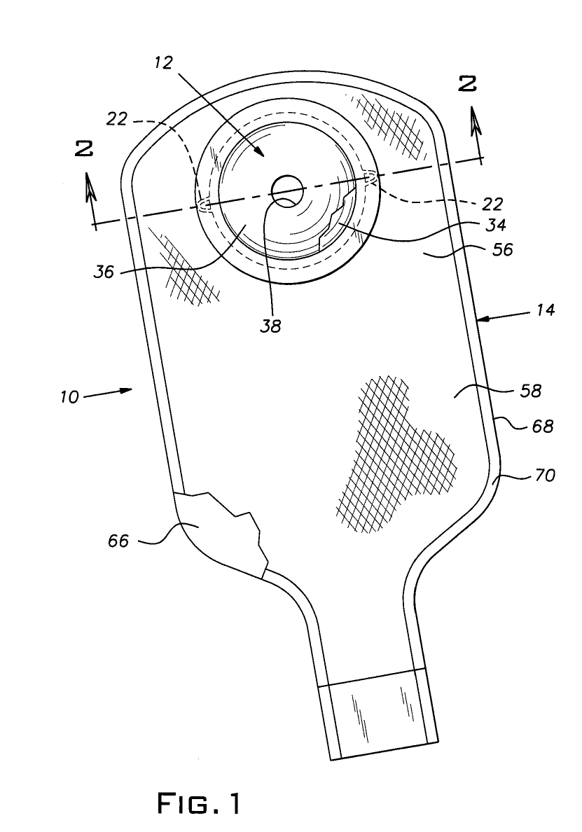

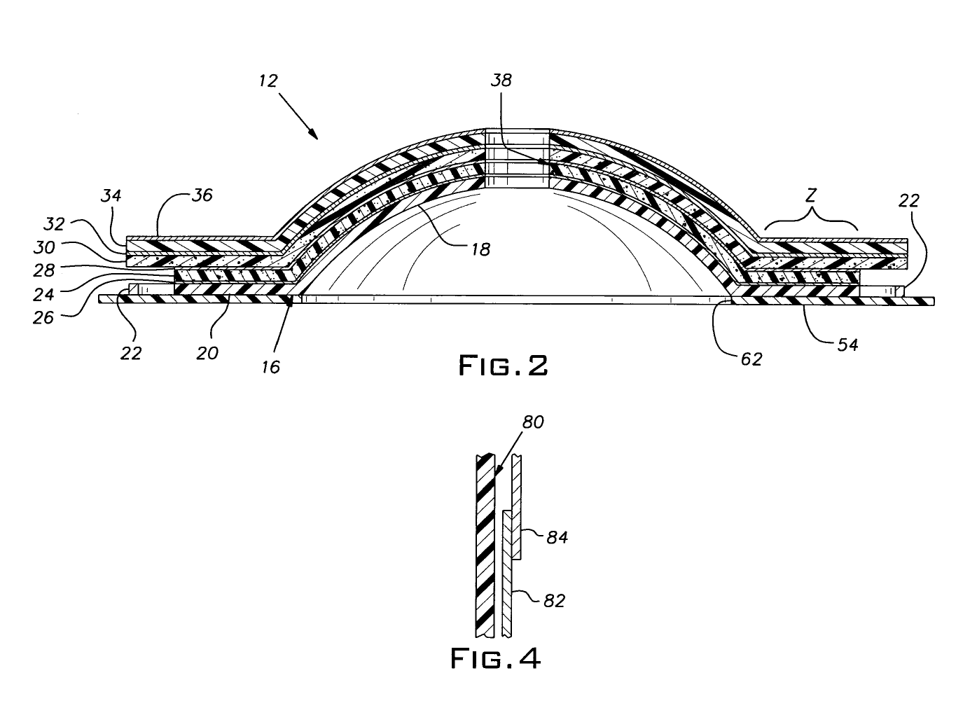

Referring now to the drawings, and particularly to FIGS. 1 and 2, there is illustrated an ostomy appliance assembly 10 according to this invention. The assembly 10 includes a mounting disc 12, adapted to be attached to a fastening belt (not shown) of an ostomate, and an ostomy pouch 14.

The mounting disc 12 includes a flexible plastic disc 16 having a convex central body portion 18 and a surrounding annular rim 20. The disc may be molded from a copolymer of ethylene and vinyl acetate (EVA). The EVA copolymer may be “ELVAX”® 450, produced by E.I. DuPont de Nemours, Inc. Wilmington, Del. The disc 16 is provided with diametrically opposed and radially extending belt mounting loops 22 at its periphery.

A first soft resilient thermoplastic foam disc 24 covers the flexible plastic disc 16. The foam disc 24 has pressure-sensitive adhesive layers 26 and 28 on each of its faces and one face is attached to the plastic disc 16. The foam disc 24, when applied to the disc 16, has an outer diameter substantially corresponding to the outer diameter of the disc 16.

A second soft, resilient thermoplastic foam disc 30 covers the first foam disc 24. The foam disc 30 has a pressure-sensitive adhesive layer 32 on one face, and the other face of the disc 24 is adhesively attached to the adhesive layer 28 of the first foam disc 24 so that the pressure-sensitive layer is exposed. The foam disc 30 has an outer diameter greater than the outer diameters of the first foam disc 24 and the disc 16. The mating loops 22 do not extend beyond the diameter of the second foam disc 30 so that the skin of the ostomate is protected from belt hooks passing through the loops 22.

An adhesive hydrocolloid skin barrier disc 34 having an outer diameter corresponding to that of the second foam disc 30 is placed on the adhesive layer 32 of the second foam disc 30. Preferably the exposed surface of the disc 34 is covered by a thin release film 36 which is removed just prior to use. Heat and pressure are applied to an annular zone z (FIG. 2) to seal the discs and to smooth the foam and hydrocolloid discs over the convex central body portion 18 of the plastic disc 16.

The adhesive skin barrier disc 34 is pliable and has both dry and wet tack. Suitable materials are Karaya-glycerine formulations or mixtures of polyacrylamide resins and other polyols and mixtures of elastomers and hydrocolloids. A stoma inlet portal 38 is provided at the center of the assembly, as illustrated in FIG. 2.

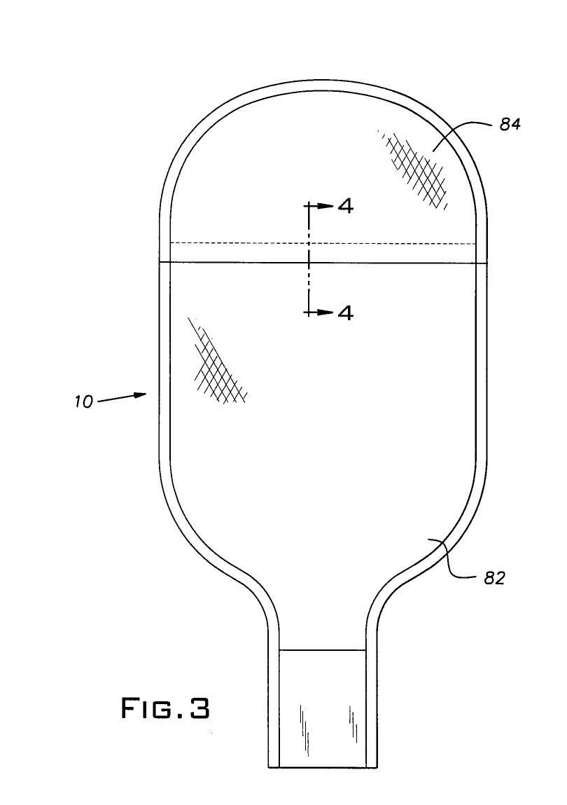

Referring now to FIGS. 3 & 4 a distal side 80 of the pouch is provided with a first opaque or translucent panel 82. The panel 82 extends from the bottom of the pouch to a level at approximately the level of the stoma opening 38 in the mounting disc 12 on the proximal side of the pouch.

A second opaque or translucent panel 84 extends from the top of the pouch and overlaps the first panel. The first and second panels 82 and 85 are heat sealed at their peripheries to the edge 86 of the distal side 80 of the pouch. Since the panels 82 and 84 are overlapped but not sealed, the user may manually separate the overlap to inspect the condition of the stoma.

What is claimed is:

1. An ostomy drainage receptacle comprising a flexible pouch defined by drainage chamber defining walls, means defining a stoma receiving opening in a first one of said walls, at least a second one of said walls being translucent or transparent, a third opaque wall sealed along a portion of its edges to said second one of said walls and a fourth opaque wall sealed along a portion of its edges to said second one of said walls and slightly overlapping an edge of said third wall.

2. An ostomy drainage receptacle according to claim 1, wherein said third and fourth walls an overlap in an area opposite said stoma receiving opening.

3. An ostomy drainage receptacle according to claim 2, wherein said overlap area is lateral.

4. An ostomy drainage receptacle according to claim 1, wherein said stoma receiving opening comprises a mounting disc, said mounting disc having a convex central body portion and a surrounding annular rim.

5. An ostomy drainage receptical according to claim 4, wherein a soft resilient foam disc covers the plastic disc.

6. An ostomy drainage receptacle comprising a flexible pouch defined by drainage chamber defining walls, means defining a stoma receiving opening in one of said walls, said means comprising a mounting disc, said mounting disc having a convex central body portion and a surrounding annular rim, a soft resilient foam disc covering said plastic disc, at least a second one of said walls being translucent or transparent, a third opaque wall sealed along a portion of its edges to said second one of said walls, an unsealed portion of an edge of said third opaque wall extending across said second of said walls, said edge being at the level of said mounting disc, a fourth opaque wall sealed along a portion of its edges to said second one of said walls, an unsealed portion of an edge of said fourth opaque wall extending across said second of said walls and overlapping said edge of said third opaque wall.
